(19) United States
(12) Patent Application Publication
(10) Pub. No.: US 2025/0160726 A1
(43) Pub. Date: May 22, 2025

(54) METHOD FOR HARMONISING DATA BETWEEN MACHINES

(71) Applicant: Genting TauRx Diagnostic Centre SDN BHD, Kuala Lumpur (MY)

(72) Inventors: Linda Sommerlade, Aberdeen Aberdeenshire (GB); Lip Tee Jin, Aberdeen Aberdeenshire (GB); Claude Michel Wischik, Aberdeen Aberdeenshire (GB); Bjöern Olaf Schelter, Aberdeen Aberdeenshire (GB)

(21) Appl. No.: 18/840,441

(22) PCT Filed: Feb. 28, 2023
(86) PCT No.: PCT/EP2023/054920

§ 371 Date: Aug. 21, 2024

(30) Foreign Application Priority Data
Mar. 3, 2022 (MY) PI2022001202

(51) Int. Cl. A61B 5/372; A61B 5/00; G16H 40/63

(52) U.S. Cl. CPC A61B 5/372; A61B 5/725; A61B 5/7257; G16H 40/63

(57) ABSTRACT

A method for harmonising data from a new machine of a first type with data from a reference machine of a second type. The method comprises receiving a new frequency domain spectrum of a process performed by the new machine and determining a harmonised new frequency domain spectrum of the process performed by the new machine by applying a set of harmonising new-machine specific weights to the new frequency domain spectrum, wherein the set of harmonising new machine-specific weights have been determined based on a filter frequency domain spectrum of a hardware filter of the new machine, and a filter frequency domain spectrum of a hardware filter of the reference machine. The harmonised new frequency domain spectrum is harmonised to a corresponding frequency domain spectrum of the process when the process is performed by the reference machine.

### FIELD OF THE INVENTION

The present invention relates to methods and systems for harmonising data between different machines. It is particularly, but not exclusively, related to methods and systems for harmonising data generated by different machines having different hardware filters, and has a particular application to EEG data.

### BACKGROUND

Electroencephalography (EEG) is a widely used imaging tool in Neuroscience, not least because of its low cost and portability. EEG is an electrophysiological monitoring technique to record electrical activity on the scalp, to determine macroscopic activity of the surface layer of the brain underneath. Typically, electrodes are placed on the scalp to measure voltage fluctuations resulting from the brain's electrical activity. EEG can be used to diagnose epilepsy, dementia, sleep disorders, tumours, strokes, depth of anaesthesia and coma, and many other conditions.

Many different EEG devices are available to cover a broad range of uses for research and routine assessments. However, it is not straightforward to compare EEG data collected from different devices. Methods optimized for one machine type might not transfer to other machine types and it becomes difficult to validate results on different data sets, as the resulting differences in the results may be much larger than the effect under investigation. The main difference between the different machine types results from the hardware filters that are used during data recording. This leads to differences between data collected from different machine types, especially in the very low and very high frequency ranges. This problem is also seen in other types of machines, and is not limited to EEG machines.

It is known that for time-domain analyses, software filters can be used to attempt comparability across different machine types. The usefulness of these filters is restricted to certain time-domain analyses and visual inspection of data. These software filters are insufficient for frequency-domain analyses involving the frequency ranges affected by the machines' hardware filters. This is because these software filters alter the power spectrum in such a way that analyses may no longer be meaningful.

The present invention has been devised in light of the above considerations.

### SUMMARY OF THE INVENTION

In general, aspects of the present invention provide methods and systems for harmonising the spectral content of data across multiple machine types, by transforming data from a new machine of a first type to be comparable to that of a reference machine of second type by applying machine-type specific weights to the spectral content data of the new machine.

According to a first aspect of the invention, there is provided a method for harmonising data from a new machine of a first type with data from a reference machine of a second type, the method comprising:
receiving a new frequency domain spectrum of a process performed by the new machine; and
determining a harmonised new frequency domain spectrum of the process performed by the new machine, the harmonised new frequency domain spectrum being harmonised to a corresponding frequency domain spectrum of the process when the process is performed by the reference machine, by applying a set of harmonising new-machine specific weights to the new frequency domain spectrum, wherein the set of harmonising new machine-specific weights have been determined based on a filter frequency domain spectrum of a hardware filter of the new machine, and a filter frequency domain spectrum of a hardware filter of the reference machine.

In this way, the harmonised new frequency domain spectrum of the process performed by the new machine is harmonised to a frequency domain spectrum of the same process performed by the reference machine, in particular by possessing the filter characteristics of the reference machine's hardware filter. Thus, data from different machines can be pooled to enable comparability of data sets recorded with different machines. Therefore, analysis results can be validated on different data sets, and data from a same experiment or process can be collected on different machines for analysis. This can increase the number of data sets used in studies, thus improving the accuracy and efficiency of such studies, and improve logistics of wide-scale studies. Furthermore, this harmonisation of the spectral content of data across different machine types is suitable for network analysis.

The simulations set out below in the detailed description demonstrate that methods according to embodiments of this aspect, and the following aspects, accurately allow for harmonisation of frequency domain data from a new machine with data from a reference machine, despite differing hardware filter characteristics.

Optional features will now be set out. These are applicable singly or in any combination with any aspect of the invention.

As used herein, a type of machine may refer to a type of hardware filter used in the machine. Thus, machines of the first type and machines of the second type may have different hardware filters. For example, machines of the first type may each have a first type of hardware filter, and machines of the second type may each have a second type of hardware filter, wherein the second type of hardware filter is different to the first type of hardware filter. Machines of the first type and machines of the second type may be manufactured by different entities, and/or collect and process data in different ways.

As used herein, a process may be any measurement and/or data collection process, wherein the data is measured/collected with a machine having a hardware filter. The process may involve measuring and/or collecting data from a subject, such as a human subject. The process may involve measuring electrical activity in the brain, e.g. an electroencephalography (EEG) process. However, the above described method for harmonising data from a new machine of a first type with data from a reference machine of a second type is not limited to EEG data; any data collected with machines that have different hardware filters may be harmonised by the methods described herein.

The set of harmonising new-machine specific weights may have been determined at least in part by dividing the filter frequency domain spectrum of the hardware filter of the reference machine by the filter frequency domain spectrum of the hardware filter of the new machine.

In some applications, the frequency domain spectrum of the hardware filter of the reference machine and/or the frequency domain spectrum of the hardware filter of the new machine may be known. In these applications, the set of harmonising new machine-specific weights may have been determined based on the known filter frequency domain spectrum of the reference machine and/or the known frequency domain spectrum of the new machine.

However, in many applications, the filter frequency domain spectra of the reference machine and/or the new machine may not be known. In these situations, the set of harmonising new machine-specific weights may have been determined based on an estimated filter frequency domain spectrum of the reference machine and/or an estimated filter frequency domain spectrum of the new machine. In particular, the filter frequency domain spectrum of the hardware filter of the new machine and/or the filter frequency domain spectrum of the hardware filter of the reference machine (upon which the set of harmonising new machine-specific weights is based) may be an estimated filter frequency domain spectrum of the hardware filter of the new machine and/or an estimated filter frequency domain spectrum of the hardware filter of the reference machine, respectively.

In particular, the estimated filter frequency domain spectrum of the hardware filter of the reference machine may be based on a set of frequency domain spectra for a plurality of different processes performed by one or more machines of the second type; and/or the estimated filter frequency domain spectrum of the hardware filter of the new machine may be based on a set of frequency domain spectra for a plurality of different processes performed by one or more machines of the first type.

The one or more machines of the first type may include the new machine. Each of the frequency domain spectra in the set corresponding to the one or more machines of the first type may be for a different process.

The one or more machines of the second type may include the reference machine. Each of the frequency domain spectra in the set corresponding to the one or more machines of the second type may be for a different process.

Preferably, the set of frequency domain spectra for a plurality of different processes performed by one or more machines of the first type comprises 30 or more frequency domain spectra. More preferably, this set comprises 40 or more, 50 or more, 100 or more, 200 or more etc. frequency domain spectra.

Similarly, the set of frequency domain spectra for a plurality of different processes performed by one or more machines of the second type comprises 30 or more frequency domain spectra. More preferably, this set comprises 40 or more, 50 or more, 100 or more, 200 or more etc. frequency domain spectra.

The present inventors have found that a set of at least 30 frequency domain spectra provides a stable approximation/estimation of the respective filter frequency domain spectrum. Increasing the number of frequency domain spectra above 30 increases the confidence further.

The filter frequency domain spectrum of the hardware filter of the new machine may be based on an average (e.g. a median) of the plurality of frequency domain spectra for the plurality of different processes performed by the one or more machines of the first type. In other words, the filter frequency domain spectrum of the hardware filter of the new machine may have been determined by taking an average (e.g. a median) of the plurality of frequency domain spectra for the plurality of different processes performed by the one or more machines of the first type.

Similarly, the filter frequency domain spectrum of the hardware filter of the reference machine may be based on an average (e.g. a median) of the plurality of frequency domain spectra for the plurality of different processes performed by the one or more machines of the second type. In other words, the filter frequency domain spectrum of the hardware filter of the reference machine may have been determined by taking an average (e.g. a median) of the plurality of frequency domain spectra for the plurality of different processes performed by the one or more machines of the second type.

In this way, an approximation/estimation of the filter frequency domain spectra of the hardware filters of the reference machine and new machine can be used as the filter frequency domain spectrum of the hardware filter of the reference machine, and the filter frequency domain spectrum of the hardware filter of the new machine, respectively, in the above method. The present inventors have found that in the harmonising weights that are applied to the new frequency domain spectrum, the common spectral properties of the plurality of different processes cancel out when the estimated filter frequency domain spectrum of the reference machine is divided by the estimated filter frequency domain spectrum of the new machine. Thus, although the approximated filter frequency domain spectra may be different from the actual (e.g. true) filter frequency domain spectra, the common spectral properties of the processes cancel out when the harmonisation weights are obtained.

Optionally, the method may include interpolating the harmonising new-machine specific weights across frequency. In this way, the harmonising new-machine specific weights are available for the same frequency bins as the new frequency domain spectrum.

The new frequency domain spectrum may be received from an external network. In some examples, the new frequency domain spectrum may be received (directly) from the new machine. Alternatively, the new frequency domain spectrum may be received from an external storage device or from local storage.

In some examples, the new frequency domain spectrum of the process performed by the new machine may be one of the set of frequency domain spectra for a plurality of different processes performed by one or more machines of the first type. In this way, the new frequency domain spectrum of the process performed by the new machine itself forms part of the set of frequency domain spectra used to calculate the estimated filter frequency domain spectrum of the hardware filter of the new machine.

Optionally, the method may comprise receiving time series data of the process performed by the new machine, and transforming the time series data into the new frequency domain spectrum of the process performed by the new machine (e.g. by Fourier filtering in an original Fourier transform).

The time series data may be received from the new machine, for example. Alternatively, it may be received from an external network, from an external storage device or from local storage.

As used herein, a frequency domain spectrum refers to a representation of one or more signals within each given frequency band over a range of frequencies. The term frequency domain spectrum may be used interchangeably herein with the term power spectrum, or the term periodogram, which is an estimator of a power spectrum. As such, the new frequency domain spectrum of the process performed by the new machine may be a new periodogram. The new periodogram may be determined by transforming time series data from the new machine into the frequency domain (e.g. by Fourier filtering), and then taking the absolute squared value of the resulting raw frequency domain spectrum.

When the method comprises transforming time series data into the new frequency domain spectrum of the process performed by the new machine in an original Fourier transform, the method may comprise retrieving harmonised time-domain data from the harmonised new frequency domain spectrum. For example, retrieving the harmonised time-domain data may include taking the inverse Fourier transform of the square root of the harmonised new frequency domain spectrum, together with the phases of the original Fourier transform of the time series data. Thus, harmonised time-domain data of the process performed by the new machine can be obtained, which is harmonised and thus comparable with time-domain data of the process when performed by the reference machine.

The harmonised time-domain data from the harmonised new frequency domain spectrum (i.e. the harmonised data from the new machine) can then be pooled with corresponding time-domain data from the reference machine. This is because this data is harmonised, and thus comparable with the reference machine data.

The data may be electroencephalographic (EEG) data. In particular, the reference machine and new machine may be EEG machines (e.g. for taking EEG measurements). However, in other embodiments, the data may be other types of data, e.g. measured by another medical monitoring/recording process.

The above method can also be applied to further machines (i.e. a second, third, fourth etc. new machine). In this way, data between multiple machines may be harmonised.

The above method may be computer-implemented. For example, the method may be implemented on one or more computer processing devices. The one or more computer processing devices may be one or more computers, servers, cloud-based devices, for example.

The method of the present aspect may include any combination of some, all or none of the above described preferred and optional features.

In a second aspect, there is provided a method for generating a set of harmonising new-machine specific weights for harmonising data from a new machine of a first type with data from a reference machine of a second type, the method comprising:
determining a set of harmonising new machine-specific weights based on a filter frequency domain spectrum of a hardware filter of the reference machine and a filter frequency domain spectrum of a hardware filter of the new machine.

Optionally, determining the set of harmonising new machine-specific weights may include dividing the filter frequency domain spectrum of the hardware filter of the reference machine by the filter frequency domain spectrum of the hardware filter of the new machine.

The method may further comprise:
receiving a set of frequency domain spectra for a plurality of different processes performed by one or more machines of the first type; and
determining the filter frequency domain spectrum of the hardware filter of the new machine based on the set of frequency domain spectra for the plurality of different processes performed by the one or more machines of the first type.

The method may further comprise:
receiving a set of frequency domain spectra for a plurality of different processes performed by one or more machines of the second type; and
determining the filter frequency domain spectrum of the hardware filter of the reference machine based on the set of frequency domain spectra for the plurality of different processes performed by the one or more machines of the second type.

The filter frequency domain spectrum of the hardware filter of the new machine may be determined, at least in part, by averaging (e.g. taking the median) of the set of frequency domain spectra for the plurality of different processes performed by the one or more machines of the first type.

The filter frequency domain spectrum of the hardware filter of the reference machine may be determined, at least in part, by averaging (e.g. taking the median) of the set of frequency domain spectra for the plurality of different processes performed by the one or more machines of the second type.

The one or more machines of the first type may include the new machine. Each of the frequency domain spectra in the set corresponding to the one or more machines of the first type may be for a different process.

The one or more machines of the second type may include the reference machine. Each of the frequency domain spectra in the set corresponding to the one or more machines of the second type may be for a different process.

Preferably, the set of frequency domain spectra for a plurality of different processes performed by one or more machines of the first type comprises 30 or more frequency domain spectra. More preferably, this set comprises 40 or more, 50 or more, 100 or more, 200 or more etc. frequency domain spectra.

Similarly, the set of frequency domain spectra for a plurality of different processes performed by one or more machines of the second type comprises 30 or more frequency domain spectra. More preferably, this set comprises 40 or more, 50 or more, 100 or more, 200 or more etc. frequency domain spectra.

The present inventors have found that a set of at least 30 frequency domain spectra provides a stable approximation/estimation of the respective filter frequency domain spectrum. Increasing the number of frequency domain spectra above 30 increases the confidence further.

The sets of frequency domain spectra may be received from an external network. In some examples, the sets of frequency domain spectra may be received from an external storage device, from local storage or directly from the one or more machines of the first/second type.

The method of the second aspect may be a computer-implemented method.

The new machine-specific weights generated in the method of the second aspect may be used as the new machine-specific weights in the method of the first aspect.

As such, according to a third aspect there is provided a method for harmonising data from a new machine of a first type with data from a reference machine of a second type, the method comprising:
receiving a set of frequency domain spectra for a plurality of different processes performed by one or more machines of the first type;
receiving a set of frequency domain spectra for a plurality of different processes performed by one or more machines of the second type;
determining a filter frequency domain spectrum of a hardware filter of the new machine by averaging the set of frequency domain spectra for the plurality of different processes performed by the one or more machines of the first type;
determining a filter frequency domain spectrum of a hardware filter of the reference machine by averaging the set of frequency domain spectra for the plurality of different processes performed by the one or more machines of the second type;
determining a set of harmonising new machine-specific weights based on the filter frequency domain spectrum of the hardware filter of the reference machine and the filter frequency domain spectrum of the hardware filter of the new machine;
receiving a new frequency domain spectrum of a process performed by the new machine; and
determining a harmonised new frequency domain spectrum of the process performed by the new machine, the harmonised new frequency domain spectrum being harmonised to a corresponding frequency domain spectrum of the process when the process is performed by the reference machine, by applying the set of harmonising new-machine specific weights to the new frequency domain spectrum.

The method of the third aspect may be a computer-implemented method.

In a fourth aspect, the invention provides a device for harmonising data from a new machine of a first type with data from a reference machine of a second type, the device comprising a processor and a memory, and wherein the memory contains machine executable instructions which, when executed on the processor, cause the processor to:
receive a new frequency domain spectrum of a process performed by the new machine; and
determine a harmonised new frequency domain spectrum of the process performed by the new machine, the harmonised new frequency domain spectrum being harmonised to a corresponding frequency domain spectrum of the process when the process is performed by the reference machine, by applying a set of harmonising new-machine specific weights to the new frequency domain spectrum, wherein the set of harmonising new machine-specific weights have been determined based on a filter frequency domain spectrum of a hardware filter of the new machine, and a filter frequency domain spectrum of a hardware filter of the reference machine.

The memory may contain machine executable instructions which, when executed on the processor, cause the processor to perform the method of the first aspect including any one, or combination insofar as they are compatible, of the optional features set out with reference thereto.

In some embodiments, the device may be a computer processing device, such as one or more computers, servers, or cloud-based devices, for example. The device may be configured to communicate via a wired and/or wireless connection with the reference device and/or the new device, and/or with one or more storage devices. In some examples, the device may form part of the new machine and/or the reference machine.

The new frequency domain spectrum of the process performed by the new machine, (and/or time series data corresponding to this frequency domain spectrum) may be obtained from an external network, directly from the new machine, or from local storage. Similarly, the set of frequency domain spectra for a plurality of different processes performed by one or more machines of the first type, and the set of frequency domain spectra for a plurality of different processes performed by one or more machines of the second type, which may be used in the determination of the filter frequency domain spectra of the hardware filters of the reference machine and the new machine respectively, may be obtained from an external network, directly from one or more machines of the first and second type respectively, and/or from local storage.

Optionally, the device may comprise one or more input/output adapters for receiving the data and/or for transmitting the harmonised data back into the network (e.g. the harmonised new frequency domain spectrum of the process performed by the new machine).

In a fifth aspect, there is provided a device for generating a set of harmonising new-machine specific weights for harmonising data from a new machine of a first type with data from a reference machine of a second type, the device comprising a processor and a memory, and wherein the memory contains machine executable instructions which, when executed on the processor, cause the processor to:
receive a set of frequency domain spectra for a plurality of different processes performed by one or more machines of the first type;
receive a set of frequency domain spectra for a plurality of different processes performed by one or more machine of the second type;
determine a filter frequency domain spectrum of a hardware filter of the new machine by averaging the set of frequency domain spectra for the plurality of different processes performed by the one or more machines of the first type;
determine a filter frequency domain spectrum of a hardware filter of the reference machine by averaging the set of frequency domain spectra for the plurality of different processes performed by the one or more machines of the second type; and
determine a set of harmonising new machine-specific weights based on the filter frequency domain spectrum of the hardware filter of the reference machine and the filter frequency domain spectrum of the hardware filter of the new machine.

The new machine-specific weights generated by the device of the fifth aspect may be used by the device of the fourth aspect as the new machine-specific weights.

The memory may contain machine executable instructions which, when executed on the processor, cause the processor to perform the method of the second aspect including any one, or combination insofar as they are compatible, of the optional features set out with reference thereto.

In a sixth aspect, there is provided a system for harmonising data from a new machine of a first type with data from a reference machine of a second type, the system comprising one or more processors and a memory, and wherein the memory contains machine executable instructions which, when executed on the processor, cause the processor to perform the method of the third aspect.

In a seventh aspect, there is provided a non-transitory computer readable storage medium containing machine executable instructions which, when executed on a processor, cause the processor to perform the method of the first aspect, the second aspect, and/or the third aspect, including any one, or any combination insofar as they are compatible, of the optional features set out with reference thereto.

Further aspects of the present invention include: a computer program comprising code which, when run on a computer, causes the computer to perform the method of the first aspect, the second aspect and/or the third aspect; a computer readable storage medium storing a computer program comprising code, which, when run on a computer, causes the computer to perform the method of the first aspect, the second aspect and/or the third aspect; and a computer system programmed to perform the method of the first aspect, second aspect and/or third aspect.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1A is a plot showing the distribution of an illustrative network measure for a healthy group and a diseased group collected on a same EEG machine;

FIG. 1B is a plot showing distributions of an illustrative network measure for a healthy group and a diseased group collected on a same EEG machine, and an illustrative network measure for the healthy group collected on a different EEG machine of a different type;

FIG. 2 is a diagram showing the results of rPDC analysis on original simulated data, filtered data and data obtained after reversing the filter effect;

FIG. 3 is a graph illustrating the comparison of a filter power spectrum reconstructed in a simulation from 100 data sets with uniformly distributed peak frequency with a known filter power spectrum;

FIG. 4 is a flow diagram of a method for harmonising data from a new machine with data from a reference machine;

FIG. 5 is a diagram illustrating an implementation of the method shown in FIG. 4;

FIG. 6 includes two graphs illustrating results of a simulation to determine approximated filter power spectra compared to a true filter power spectrum;

FIG. 7 is a graph showing a comparison of a harmonisation curve (set of weights) obtained from a set of estimated power spectra with a true harmonisation curve derived from known filter power spectra;

FIG. 8A is a graph of the approximate filter value (e.g. median periodogram) at 10 Hz with error bars for increasing number of data, obtained from a simulation process;

FIG. 8B is a graph of the approximate filter value (e.g. median periodogram) at 10 Hz with error bars for increasing number of data sets, obtained from EEG data;

FIG. 9 is a graph of the approximate filter value (e.g. median periodogram) at 10 Hz with error bars for 50 repetitions of randomly selecting 30 out of 190 power spectra, obtained from EEG data;

FIG. 10 includes two plots of power spectra of filters of a new machine and a reference machine, obtained from a simulation process;

FIG. 11 is a graph of a theoretical harmonisation curve obtained in a simulation process;

FIG. 12 includes two plots of approximated power spectra of filters of a new machine and a reference machine, obtained in a data harmonisation method;

FIG. 13 is a plot comparing theoretical weights corresponding to the harmonisation curve in FIG. 11, to the weights derived from the approximate power spectra in FIG. 12;

FIG. 14 is a diagram showing the results of rPDC analysis on original simulated data from a new machine, reference machine, and the new machine harmonised to the reference machine;

FIG. 15 is a graph of the rPDC values obtained from simulation of a new machine type, reference machine type and harmonised reference machine type;

FIG. 16 includes two plots of rPDC data, for comparison of results with a data harmonisation process, to results without the data harmonisation process, such as that of FIG. 4;

FIG. 17 is a plot showing distributions of an illustrative network measure for a healthy group and a diseased group collected on a same EEG machine, and an illustrative network measure for the healthy group collected on a different machine of a different machine type, wherein the distributions for the healthy group have been harmonised according to a data harmonisation process, such as that of FIG. 4;

FIG. 18 is a diagram of Grand Average ERPs from a new machine used to collect EEG data for an ERP experiment; and

FIG. 19 is a diagram of Grand Average ERPs from the new machine used to collect the EEG data for the ERP experiment, with the data harmonised to a reference machine.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

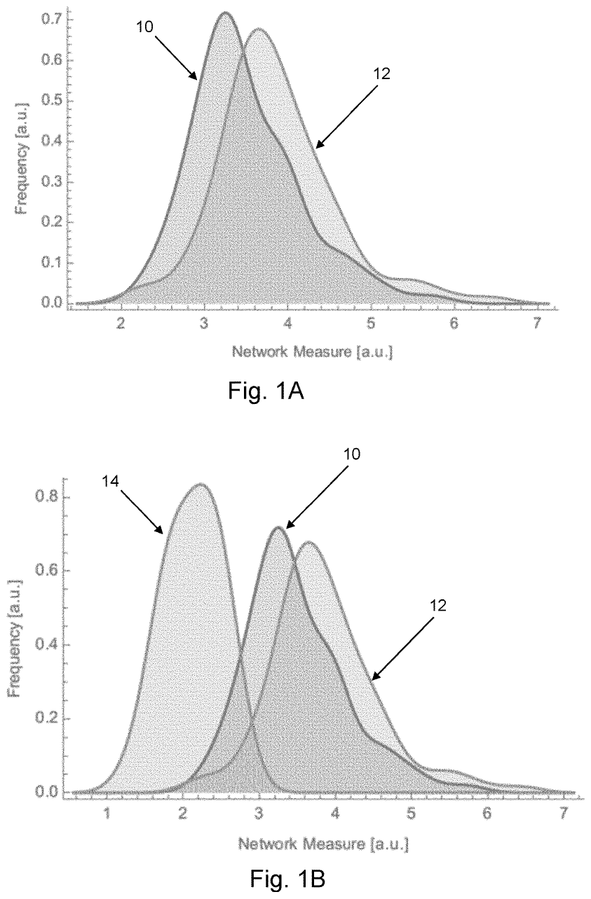

FIG. 1A is a plot showing how, for a single machine type (machine type 1), an illustrative EEG network measure is different at the group level for a healthy group 10 compared to a diseased group 12. In this illustrative example, the network measured used is frontal-out-degree, which is based on a directed measure and measures the average connectivity strength going out from frontal electrodes.

However, when the same analysis is applied to EEG recording with a different machine type (machine type 2), the network measure is different. This is illustrated in FIG. 1B, which shows the same fontal-out-degree network measure for the healthy group 10 and the diseased group 12 as measured by machine type 1, but where the distribution of the network measure for the healthy group 14 as recorded with machine type 2 is also shown. As shown in FIG. 1B, the distribution of the network measure for the healthy group as measured by machine type 1 and machine type 2 is different (i.e. plot 10 is different to plot 14). In particular, in this example, the difference between two EEG machines is larger than the disease effect. This difference highlights how it is not possible to directly compare derived EEG measures from different EEG hardware.

Data, such as EEG data, from both a new machine (e.g. of machine type 2) and a reference machine (e.g. of machine type 1) will have been subject to the specific hardware filters of the machine type, each with their own characteristics. As such, the power spectrum of a hardware-filtered process (the “joint power spectrum”) comprises the power spectrum of the process and the power spectrum of the hardware filter (and in particular, the product of the power spectrum of the process and the power spectrum of the hardware filter). It is the different power spectra of the hardware filters which result in the difference between distributions of a network measure for a same group, when the data is from different machine types.

To harmonise data across different machine types in the frequency domain, the present inventors have found that harmonising weights can be applied to the power spectrum of a hardware-filtered process. The harmonising weights are obtained by dividing the power spectrum of a reference machine's hardware filter, by the power spectrum of the new machine's hardware filter. The power spectrum of the hardware-filtered process performed by the new machine is then transformed by multiplying it with the obtained harmonising weights. This harmonised power spectrum is then harmonised to possess the filter characteristics of the reference machine's hardware filter. After the harmonising weights are applied, the harmonised data in the time domain can be retrieved from the harmonised power spectrum as long as the original data was received in the time domain such that the phases of an original Fourier transform of the non-harmonised data into the frequency domain are available, as set out below.

The present inventors carried out simulations to demonstrate that it is possible to reverse the effect of a hardware filter on simulated data. This is first demonstrated using simulated data confined to a single machine type and a known filter power spectrum. In particular, if the filter power spectrum is known, its effect on the data can be reversed by dividing the joint power spectrum (e.g. the power spectrum of the underlying process multiplied by the power spectrum of the hardware filter) by the filter power spectrum.

This process of reversing the filter effect preserves frequency domain measures such as renormalised partial directed coherence (rPDC). This was demonstrated using simulated data to show how hardware filters affect rPDC values of a connection, as set out below.

A 3-dimensional vector autoregressive process (VAR) with a model order of 2, VAR[2] was simulated, and a filter with known characteristics was applied to each of the three individual processes.

The vector autoregressive process was defined by Equation 1:

$$\vec{x} ( t ) = {\sum }_{r = 1}^{2} {α}_{r} \vec{x} ( t - r ) + {∈}_{x} ( t ) \qquad ( 1 )$$

$$with$$

$${α}_{1} = ( \begin{matrix}1.6 & 0 & 0 \\ 0.3 & 1.5 & 0 \\ 0 & 0.5 & 1.7\end{matrix} ) , {α}_{2} = ( \begin{matrix}- 0.8 & 0 & 0 \\ 0 & - 0.8 & 0 \\ 0 & 0 & - 0.8\end{matrix} ) .$$

The three-dimensional VAR process was simulated with N=20,000 data points per channel. The 20,000 data points were chosen to represent 100 seconds of data collected with a sampling rate of 200 Hz.

The interaction structure of this system comprises three processes, where process x₁ influences process x₂ which in turn influences process x₃. The influence of process x₁ onto process x₃ is indirect since it is mediated by process x₂.

A filter was then simulated by applying an AR[1] process in the time-domain to each time-series of the simulated data.

This AR[1] process is defined in Equation 2:

$$Y ( t ) = bY ( t - 1 ) + x ( t ) \qquad ( 2 )$$

where x(t) is each of \vec{x}(t) in Equation 1 (taken separately). Y(0) was set to 0.

The analytical solution of the power spectrum of this known filter is defined by Equation 3:

$${S}_{filter} ( f ) = \frac{1}{{( 1 - b \cdot {e}^{- 2 π * i * f} )}^{2}} \qquad ( 3 )$$

The value of b in Equation 3 defines the shape of the filter characteristic and was set to 0.9 in the simulation, and is defined in Equation 2 above.

The first step in the simulation process was estimating a power spectrum, by calculating a periodogram, for each dimension of the simulated data. To do this, the time series data was Fourier transformed to provide raw data Fourier transform, values for positive frequencies were extracted, and the absolute square of the raw data Fourier transform was taken, which is the periodogram. The filter spectrum was then calculated for each frequency bin present in the periodogram using Equation 3 above. Each frequency bin of the periodogram was then divided for each dimension by the value of the filter spectrum at the respective frequency. With these steps, the filter effect on the periodogram is reversed and the result is a “corrected periodogram”.

In order to obtain the time-domain data corresponding to the corrected periodogram, the absolute value of the corrected periodogram and the phases of the raw data Fourier transform were used in an inverse Fourier transform.

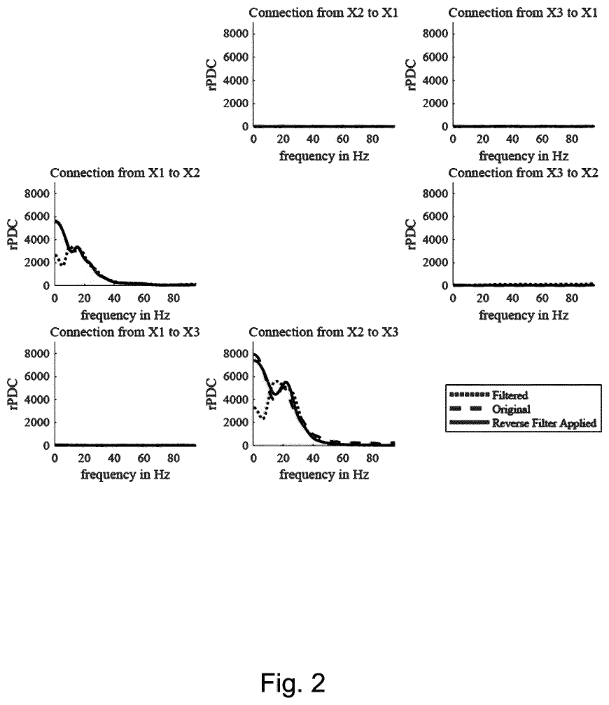

An rPDC analysis performed on original simulated data and filtered simulated data was carried out, as well as the rPDC analysis on the simulated data obtained after reversing the filter effect. FIG. 2 shows the results of rPDC analysis on the original simulated data, filtered data and data obtained after reversing the filter effect. FIG. 2 illustrates that the curves for the data obtained after reversing the filter effect almost completely overlap with the curves from the original simulated data, indicating that by dividing the joint power spectrum by the power spectrum of the filter, the filter effect can be reversed. Thus, if the power spectra of the hardware filters are known, it is possible to reverse the effect of the filters and thus pool data from different machines with different hardware filters.

However, for most applications, the filter power spectrum is not known. If the process is a white noise process, the power spectrum of the filter may be inferred as the power spectrum of the white noise process is constant across frequencies. Similarly, if there are a plurality of power spectra for different processes filtered by the same filter, and these processes have uniformly distributed spectral peaks, a good approximation of the filter spectrum may still be inferred by taking the average (e.g. the median) of the estimated spectra. This is because the different spectral peaks may cancel each other out when the average (e.g. the median) is taken, and what remains is the filter power spectrum which is common to all filtered processes.

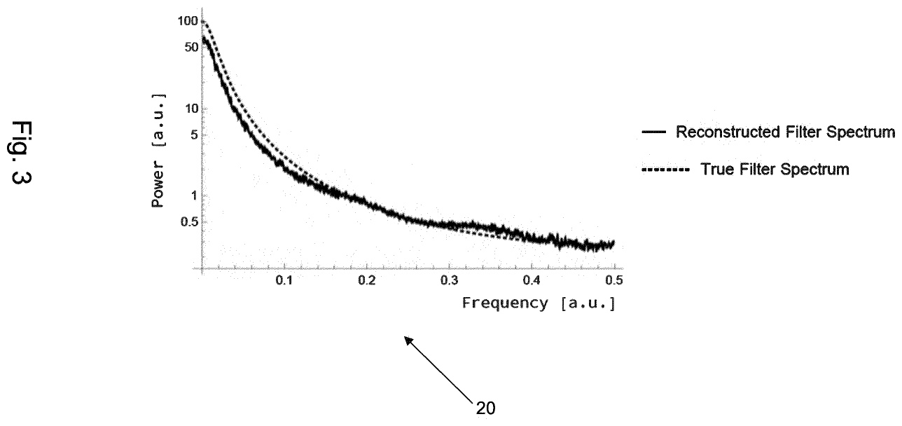

The present inventors have demonstrated this by simulating 100 univariate autoregressive processes of order two (AR[2]) with peak frequencies uniformly distributed across the whole frequency range. The AR[1] process above was used to simulate a filter according to Equation 3. The median of the spectral estimates (e.g. smooth periodograms) at each frequency were then taken and compared to the known filter power spectrum. The results are shown in the plot 20 of FIG. 3. FIG. 3 shows how there is good agreement between the median of the spectral estimates and the known filter power spectrum, confirming that the filter can be reconstructed from data if a set of many power spectra with uniformly distributed peak frequencies are available.

However, for many applications, the data available may not include a white noise process, or many power spectra with same filter (e.g. from a single machine type) that contain uniformly distributed spectral peaks. Instead, data may be available from different processes, wherein the processes share some spectral characteristics (e.g. all have a peak in a certain frequency range). Therefore, it is more difficult to reconstruct the filter power spectrum by taking the median of the estimated spectra, because the different spectral peaks do not fully cancel each other out when the median is taken. The result of taking the median is therefore a combination of the filter power spectrum and the common spectral properties of the process. The present inventors have found that increasing the number of power spectra in the data set can help reduce this issue only if the additional power spectra broaden the distribution of spectral peaks. Nevertheless, in many applications, the aim is to harmonise data from the same process, or similar processes, recorded with different machines. Thus, the approximated filter power spectra from both the new machine and the reference machine, will contain the common spectral properties of the processes.

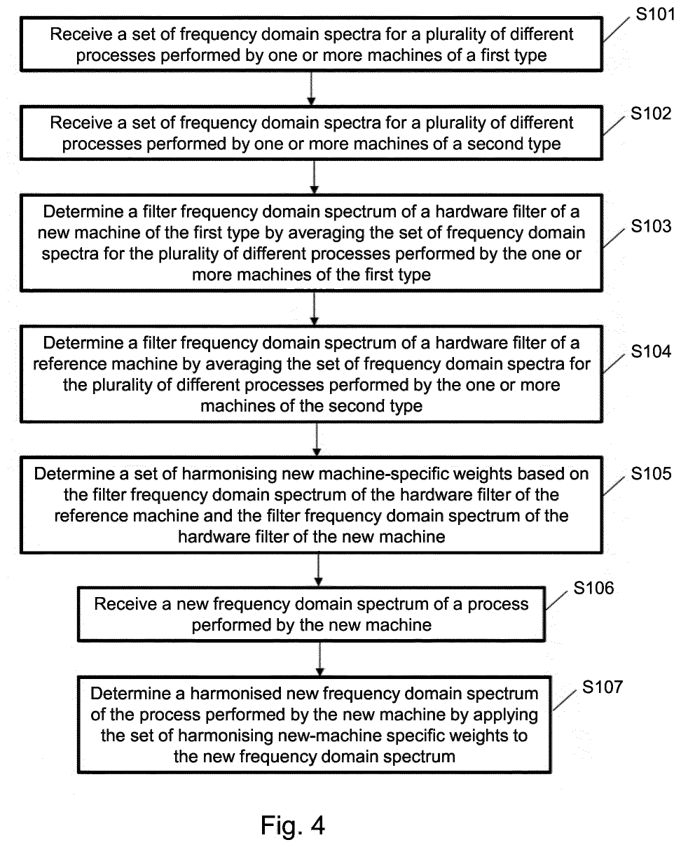

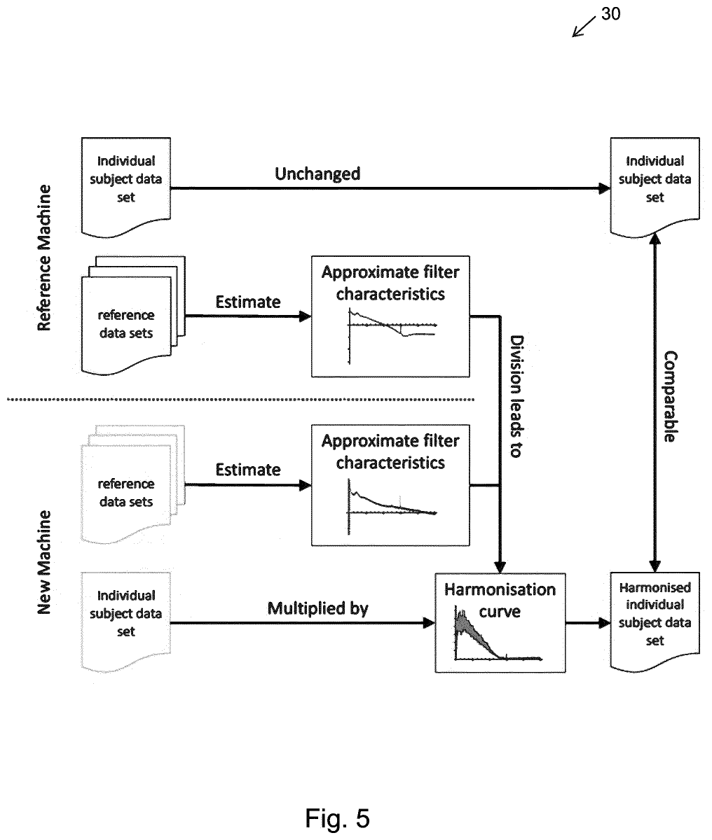

FIG. 4 is a flow diagram of an implementation of a method for harmonising data from a new machine with data from a reference machine. FIG. 5 is a diagram 30 also illustrating this method. The methods illustrated in FIG. 4 and FIG. 5 may be performed at a computing device, for example.

At S101 of FIG. 4, a set of frequency domain spectra for a plurality of different processes performed by one or more machines of the first type is received. This set of frequency domain spectra may be received from an external network, or from local storage, for example. In FIG. 5, the set of power spectra are labelled “reference data sets” for the “New Machine”.

At S102 of FIG. 4, a set of frequency domain spectra for a plurality of different processes performed by one or more machines of the second type is received. Again, this set of frequency domain spectra may be received from an external network, or from local storage, for example. Again, in FIG. 5, the set of power spectra are labelled “reference data sets” for the “Reference Machine”.

S101 and S102 may be performed in any order or simultaneously.

At S103 of FIG. 4, a filter frequency domain spectrum of a hardware filter of the new machine is estimated by averaging (e.g. taking the median of) the set of frequency domain spectra for the plurality of different processes performed by the one or more machines of the first type.

Similarly, at S104 of FIG. 4, a filter frequency domain spectrum of a hardware filter of the reference machine is estimated by averaging (e.g. taking the median of) the set of frequency domain spectra for the plurality of different processes performed by the one or more machines of the second type.

For completeness, these determined filter power spectra (labelled “Approximate filter characteristics” in FIG. 5) of both the reference machine and the new machine are approximations. In reality, they may be different to the true filter power spectra. However, this difference is accounted for when the weights are calculated later in the method herein described.

S103 and S104 may be performed in any order or simultaneously.

At S105 of FIG. 4, a set of harmonising new machine-specific weights are determined based on the filter frequency domain spectrum of the hardware filter of the reference machine and the filter frequency domain spectrum of the hardware filter of the new machine. This set of harmonising new machine-specific weights is labelled as “Harmonisation curve” in FIG. 5. The set of weights are calculated by dividing the approximated filter power spectrum of the reference machine with the approximated filter power spectrum of the new machine. The common spectral properties of the plurality of different processes cancel out when the approximated reference hardware frequency domain spectrum is divided by the approximated new hardware frequency domain spectrum. Thus, although the approximated filter frequency domain spectra may be very different from the actual (e.g. true) filter spectra, the common spectral properties of the processes cancel out when the harmonising weights are calculated.

At S106, a new power spectrum of a process performed by the new machine is received. The new power spectrum of the process performed by the new machine may be received from an external network, or from local storage, for example.

In some examples, the new power spectrum of the process performed by the new machine may be obtained from time series data of the process performed by the new machine. In these examples, the method may comprise receiving time series data of the process performed by the new machine, and transforming the time series data into the new frequency domain spectrum of the process performed by the new machine in an original Fourier transform. The time series data may be received from an external network, or from local storage. Once the new power spectrum of the process performed by the new machine is obtained, it may be stored locally such that it can then be received in S106.

For completeness, in some examples, the new power spectrum of the process performed by the new machine may be one of the frequency domain spectra in the set of frequency domain spectra received at S101 (e.g. one of the frequency domain spectra for a plurality of different processes performed by one or more machines of the first type).

At S107, the weights are applied to a new power spectrum of a process performed by the new machine (labelled “Individual subject data set” for the New Machine in FIG. 5) to generate a harmonised new power spectrum (labelled “Harmonised individual subject data set” in FIG. 5). The harmonised new power spectrum for the new machine is comparable with a power spectrum of the process when the same process is performed by the reference machine, because the effects of the hardware filters of the new machine and the reference machine has been harmonised (see FIG. 5). As such, this data can be pooled for further analysis. If necessary, the weights may be interpolated across frequency, such that weights are available for the same frequency bins as the new power spectrum.

Optionally, when the new power spectrum of the process performed by the new machine has been obtained from time series data, the method may also comprise transforming the harmonised new power spectrum into time-domain data (by taking the inverse Fourier Transform of the square root of the harmonised new power spectrum together with the phases of the original Fourier transform of the time series data). Then, time-domain data can be directly compared with time-domain data of the reference machine.

The method may only comprise S106 and S107 of FIG. 4 (e.g. if the set of harmonising new machine-specific weights are already known). A method for generating the set of harmonising new machine-specific weights is set out in S101-S105 of FIG. 4.

As mentioned above, the determined filter power spectra of both the reference machine and the new machine are only approximations. In reality, they may be different to the true filter power spectra of these filters. This is illustrated in FIG. 6, which shows two graphs 40, 42 illustrating a simulation of 100 AR[2] processes with peak frequency varying in a fixed frequency range. FIG. 6 illustrates the results of a simulation in which two different AR[1] processes (Equation 3 above, with b=0.9, and b=0.2, in graph 40 and 42 respectively of FIG. 6) to simulate two different filters are applied to 50 time series each (i.e. recordings performed on two different machine types). The resulting filter approximations are poor as they also contain the frequency content common to all processes represented in the set of power spectra used to calculate the approximation.

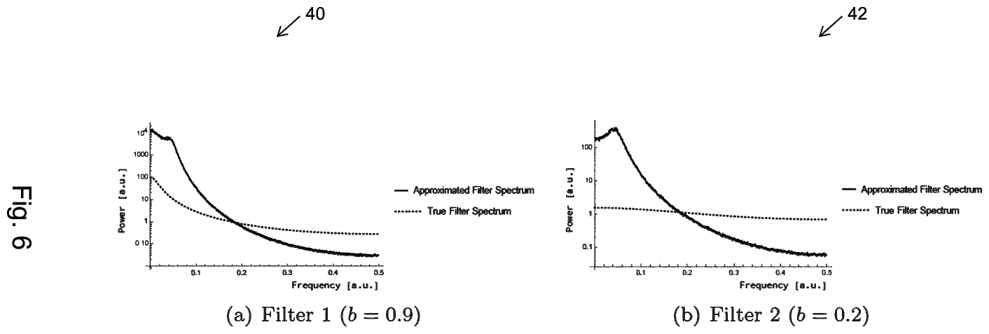

However, when the weights for filter harmonisation are calculated, the common spectral properties cancel each other out. This is illustrated by the graph 50 shown in FIG. 7, in which a comparison of weights across frequency derived from data with those calculated from the known true filter spectra are shown. This may also be defined as a “harmonisation curve”.

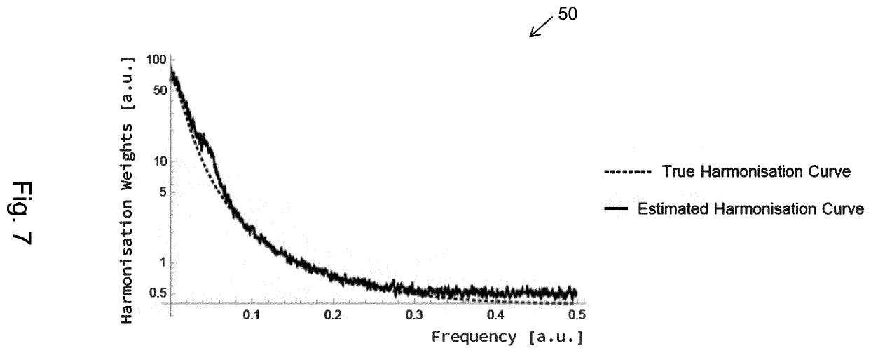

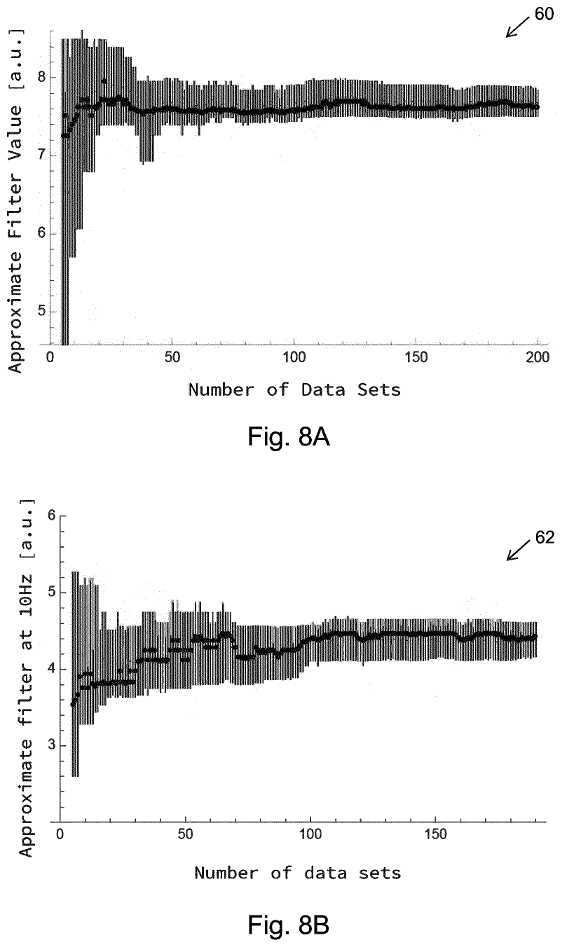

The present inventors have found that it is preferable if the set of power spectra for a plurality of different processes performed by one or more machines of the same type as the reference machine and the set of power spectra for a plurality of different processes performed by one or more machines of the same type as the new machine, each comprise at least 30 power spectra. In particular, the present inventors used an AR[2] process with peak frequency in a specific frequency range to investigate how many power spectra are needed to obtain a stable approximation of the filter power spectra. The median of the estimated spectra (the smoother periodograms with a smoothing width of 20 frequency bins) of 5 power spectra were taken, and then the number of power spectra was increased up to 200. FIG. 8A shows a graph 60 of the resulting median with error bars for one frequency. It can be observed that for very few power spectra in the set, the approximate filter value still varies and has large confidence intervals indicating it is not well defined. From a set of around 30 power spectra, it becomes stable and increasing the number of data sets increases the confidence. Thus, the present inventors concluded that a minimum of 30 power spectra in the data set is preferable.

This analysis was also repeated with EEG data measured with one EEG machine type to demonstrate that at least 30 power spectra in the data set is preferable. The analysis was based on data collected from 83 healthy subjects and 107 diseased subjects. A number of power spectra in the set for determining the filter power spectrum were randomly selected from the pool of the 190 available. As in the simulation, the analysis was started with 5 power spectra in the data set and increased up to the available maximum of 190. FIG. 8B shows a plot 62 of the obtained value for the approximate filter power spectrum at 10 Hz over the number of power spectra used in the data set for the approximation. Similarly to in the simulation results in FIG. 8A, from around 30 power spectra in the data set, the value obtained for the approximate filter power spectrum becomes stable.

For completeness, the inventors also explored if the approximation is similar when different power spectra are used for the approximation. They set the number of power spectra in the data set to 30 and randomly chose from the available 190 power spectra 50 times. The results are shown in graph 64 in FIG. 9 for the approximate filter value at 10 Hz. Only small variations are seen, and therefore it is established that 30 power spectra in the data set are sufficient to obtain a stable and reproducible approximation of the filter power spectrum. More power spectra in the data set are desirable but not required.

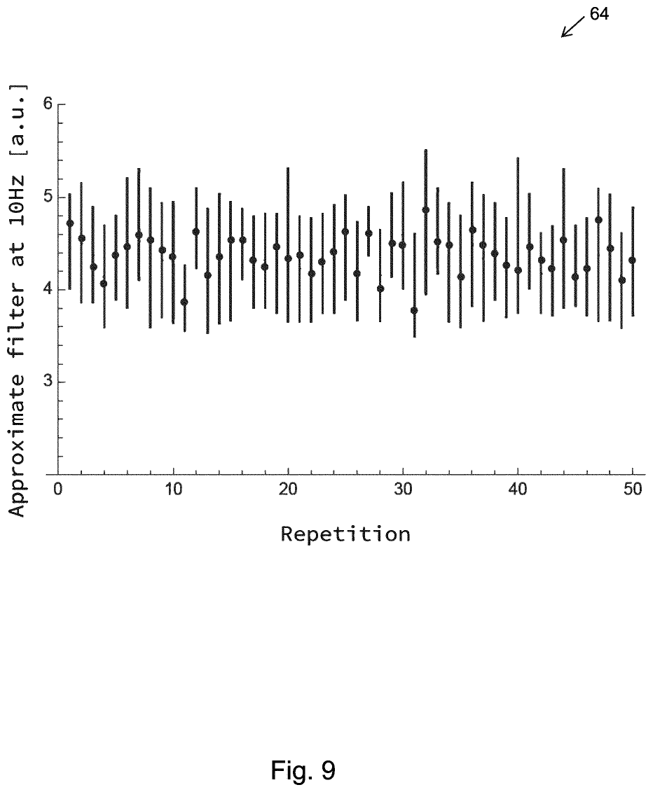

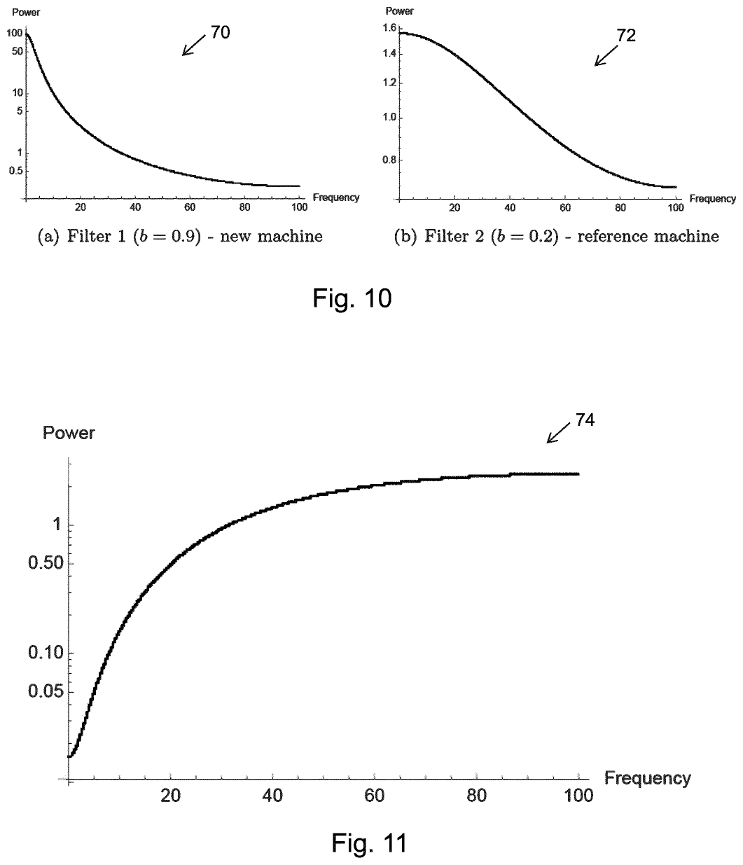

The present inventors tested the above disclosed method of harmonising data from a new machine with a reference machine with simulations. The same three-dimensional system as described above (i.e. as defined by Equation 1). Two different filters were applied to the same realisations of the system to produce data for the reference machine and data for the new machine. The power spectra of the two different filters were applied to the same realisation of the three-dimensional system as defined above in Equation 3, with b=0.9 for the new machine and b=0.2 for the reference machine. FIG. 10 shows plots 70, 72 of the power spectra of the resulting filters for the new machine and the reference machine, respectively. Both spectra are plotted on a logarithmic scale.

When the power spectrum of the filter from the reference machine is divided by the power spectrum of the filter from the new machine, the theoretical weights for harmonising data are obtained. This theoretical harmonisation curve is shown in graph 74 of FIG. 11. However, as discussed above, in most application the power spectra of the filters are unknown and thus the harmonisation curve has to be estimated.

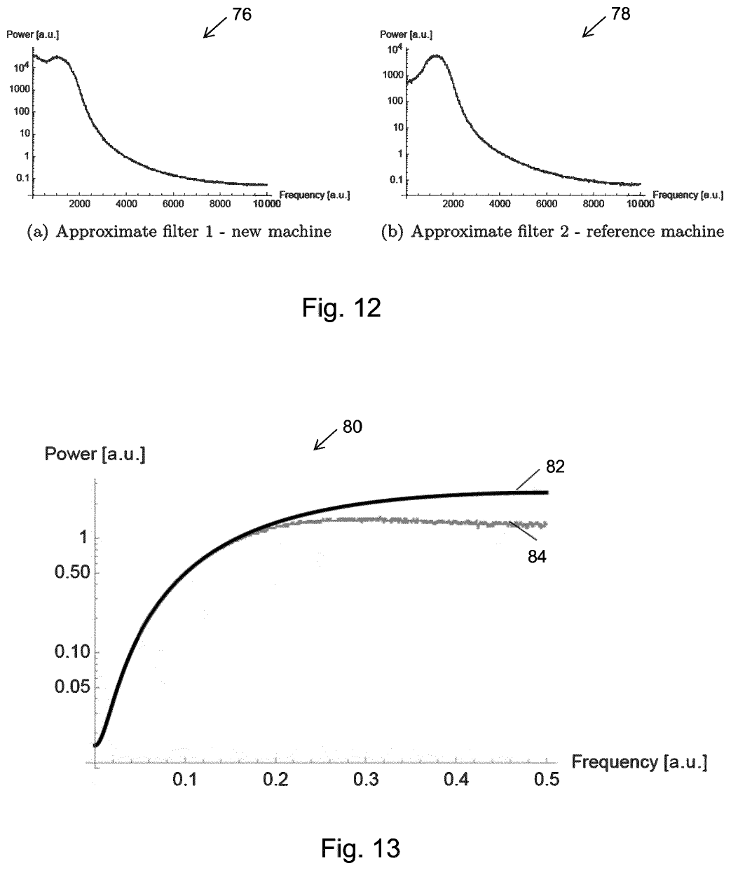

To demonstrate how to approximate the power spectra of filters, the present inventors separately simulated 10 three dimensional systems using the same VAR coefficients as described above in relation to Equation 1 and applied the simulated filters to the time series for both the reference machine and the new machine. This corresponds to obtaining 10 data sets from the same subject with both machines at the same time which can then be used to approximate the filter power spectra. The simulation of 10 realisations of a three-dimensional system results in 30 estimated spectra (e.g. smoothed periodograms) for each of the two machines. A smoothing width of 20 frequency bins was used. The median of the 30 estimated spectra (10 realisation of three dimensions each) was taken for the approximated power spectra of the filters. FIG. 12 shows plots 76, 78 of the approximated power spectra of the new machine and the reference machine, respectively. The approximated power spectra contain a combination of the respective filter power spectrum and the common spectral properties of the processes from which they are derived. Through the division of the approximated power spectrum of the reference machine by that of the new machine, the common spectral properties of the processes cancel out and the weights to be applied to the joint power spectrum of the new machine are obtained.

FIG. 13 shows a plot 80 of the resulting theoretical weights 82 and the weights derived from the estimated power spectra 84. As shown, the weights derived from the estimated power spectra closely match the theoretical weights.

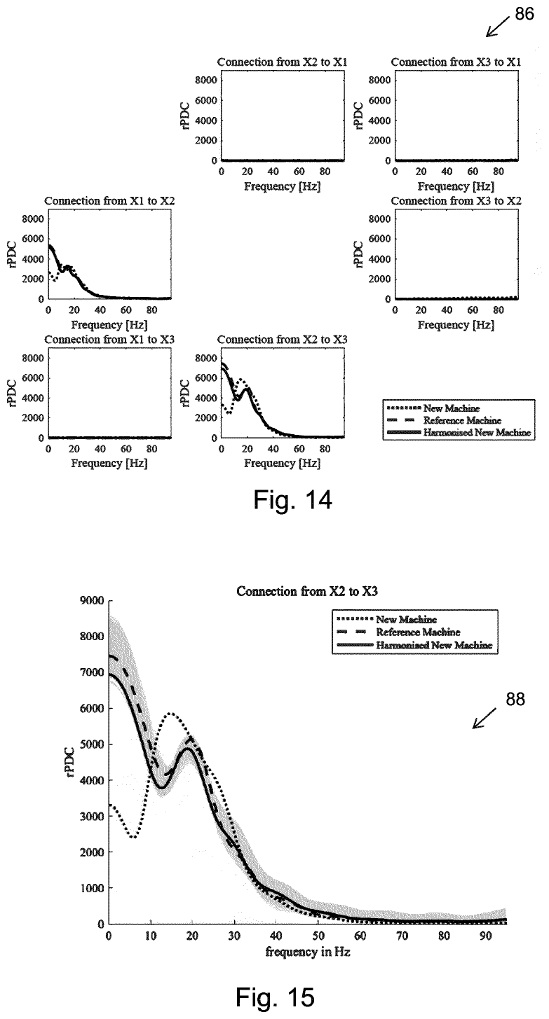

An rPDC measure was also used to compare connection strengths between the processes from the reference machine, new machine and new machine harmonised to the reference machine. FIG. 14 shows a diagram 86 of the results obtained from the rPDC analysis. For all three machine scenarios (e.g. reference machine, new machine and new machine harmonised to reference machine), the analysis only shows non-zero rPDC values for the connections present in the simulation. For the present connections (x₁ to x₂ and x₂ to x₃), it can be seen that the values obtained from the new machine largely differ from those of the reference machine. The reason for this difference is the different filters, because the same original simulated data is used for this analysis. After applying the above harmonisation procedure to the data from the new machine, the resulting values are comparable to those obtained from the reference machine. FIG. 14 shows how the above-described method can be used to obtain comparable connection strengths, because the rPDC values for the new machine harmonised to the reference machine, match those of the reference machine.

A further 500 realisations of the three-dimensional system (e.g. as defined by Equation 1) were simulated, with the reference filter applied to each time series. These 500 realisations were then simulated to produce confidence bands for the rPDC values. The rPDC values obtained from the harmonised new machine to the band of rPDC values for the reference machine. FIG. 15 shows a plot 88 of the rPDC values obtained from the simulation for the connection from x₂ to x₃. The plot 88 in FIG. 15 shows there is a large difference between the reference machine results (grey lines and dashed line) and the new machine results (dotted line). The harmonised new machine results (solid black lines) can also be compared to the reference machine results (grey lines and dashed line). FIG. 15 shows that the harmonised new machine results overlap with the reference machine results, confirming that the above described procedure is effective in obtaining comparable connection strengths.

FIG. 16 shows two graphs; the first graph 90 shows rPDC results for the new machine and the reference machine with their different filters; and the second graph 92 shows the rPDC results after harmonisation. In the simulation, the connection strength for system A was chosen to be higher than for system B. Graph 90 shows that the rPDC values for the system are higher in the low frequency for system B whereas system A has higher rPDC values for higher frequencies; thus indicating how the connection strength is incorrect at low frequencies where no harmonisation is performed. In contrast, graph 92 shows the rPDC values for system A are higher than those for system B throughout, correctly indicating the system A has the stronger connection, and confirming that the above described harmonisation procedure is effective in obtaining comparable connection strengths even when the systems have different hardware filters. It further illustrates that even if it is not possible to reverse the filter effect the relative connection strengths can be retrieved by the above described harmonisation procedure.

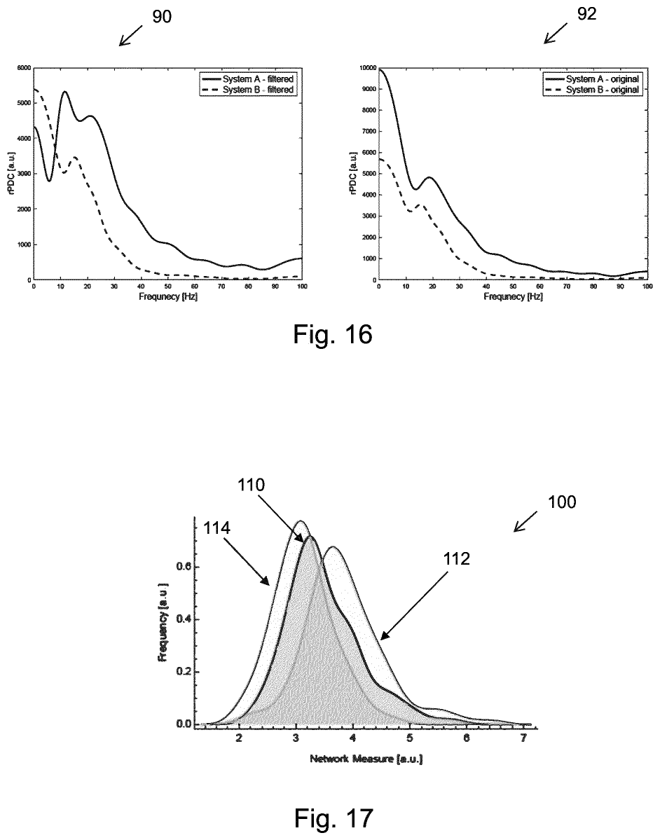

As mentioned above, the present inventors also performed analysis with EEG data. FIG. 17 shows a corresponding plot 100 to that shown in FIG. 1B, but with the above described harmonisation method applied to the data measured with machine type 2. Plot 100 shows that the distribution of the network measure obtained from the healthy group 114 measured with machine type 2 overlaps with the distribution of the network measure obtained from the healthy group 110 measured with machine type 1 (corresponding to curve 10 in the plot of FIG. 1B). The diseased group 112 measured with machine type 1 (corresponding to curve 12 in the plot of FIG. 1B) is also shown. In other words, the results from the two different machine types for the healthy groups 110, 114 becomes comparable after applying the above-described harmonisation method.

To verify that the above-described method would also be effective when the frequency-domain results are transformed into the time-domain, the present inventors applied the above described method to data obtained from an experiment that investigates event-related potentials, a known time-domain analysis of EEG data. An event-related potential (ERP) is the measured brain response that is the direct result of a specific sensory, cognitive or motor event. The data from an ERP experiment, that has shown differences in two different stimuli, was used to test that the application of the above described proposed harmonisation method does not disrupt the conclusions drawn from analysis of event-related potentials.

The ERP experiment investigates the standard old/new effect (HITs vs Correct Rejections), where subjects are presented with stimuli and asked to judge if the stimulus is part of a studied list (i.e. old) or not (i.e. new). Throughout the ERP experiment, EEG was recorded continuously with 64 electrodes on the scalp while subjects were performing the tasks. Subjects were shown objects appearing in various locations on a screen in an encoding phase. Afterwards, during a retrieval phase, subjects were shown the same objects and additional random objects, that were not previously shown (i.e. new objects). There were 12 object-location encoding phases each followed by a retrieval phase. Each encoding phase consisted of 12 trials. In addition to the 12 old objects shown during the encoding phase, 12 new objects were included in each retrieval phase. Therefore, each retrieval phase consisted of 24 trials. This amounted to 12×24=288 trials for each subject. During the retrieval phase, subjects were required to press a button to indicate where the objects had appeared during the encoding phase. An additional button could be pressed by the subject if they recognised the object as one that was not shown during the encoding phase (i.e. a new object). During the retrieval phase, if the subject correctly assigned the location in which the object had appeared during the encoding phase, the trial was considered to be a “HIT”. If the subject correctly recognised that the object is a new object that did not appear in the encoding phase, the trial was considered as a “Correct Rejection” (CR). EEG data for this ERP experiment was recorded from 59 healthy subjects.

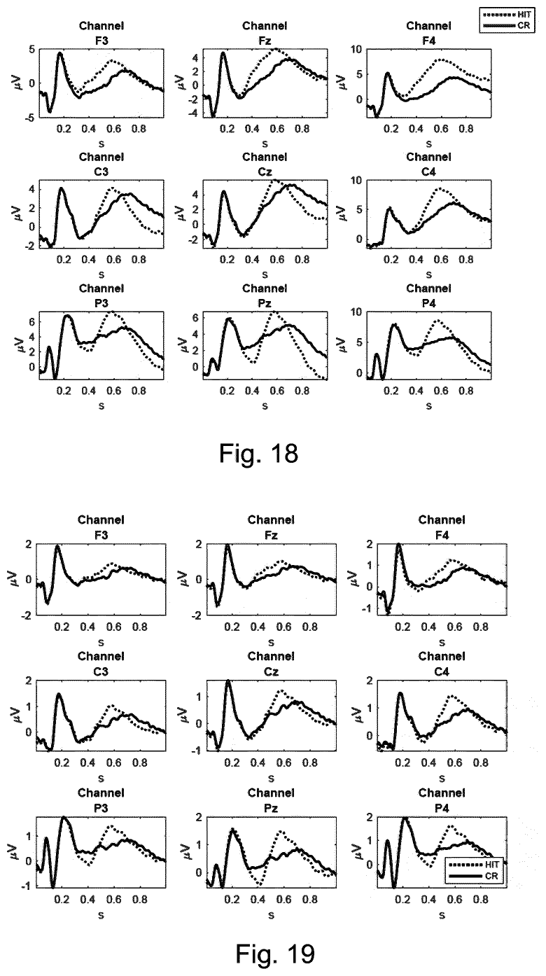

The following standard ERP pre-processing was applied to the continuous EEG data recorded during the ERP experiment. The continuous data is first bandpass filtered between 0.1 Hz and 35 Hz. The data was then referenced to the average reference and cut into epochs based on the trials. The epoch for each trial contained the EEG time series from the time the stimulus was shown to the subject (0 s) to one second after the stimulus was shown to the subject (1 s). Subjects with fewer than 16 trials for either HIT or CR condition were excluded from the analysis to ensure sufficient data is available for averaging. This resulted in 6 of the 59 subjects being excluded from the analysis. The amplitudes of all trials from all subjects were averaged to produce a grand average for each condition. The resulting ERP grand averages, shown in FIG. 18, shows a clear difference between the HIT and CR conditions from 0.4 s to 0.7 s.

To demonstrate that the above described harmonisation method does not disrupt the ERPs, the procedure above was repeated after applying the harmonisation method to the data. 190 data sets recorded with a reference machine were used to obtain the approximated filter power spectrum of the reference machine. The filter power spectrum of the new machine was approximated based on data from the 59 subjects who participated in the ERP experiment. However, data from a resting state segment that preceded the ERP experiment was used to obtain the approximated filter power spectrum of the new machine. The harmonised data was then analysed in the same way as the original data. The resulting ERP grand averages are shown in FIG. 19.

It can be seen from FIG. 18 and FIG. 19 that in both cases, the two conditions (HIT and CR) are well separated. Furthermore, by comparing the y-axis, after the harmonisation (as shown in FIG. 19), the amplitudes of the ERP grand averages are lower. Different amplitudes may be caused by different EEG machine types having different amplification. This can be corrected by amplifying the grand average ERPs with a fixed factor. The results in FIG. 18 and FIG. 19 are similar, indicating that the harmonisation method disclosed herein does not disrupt the grand average ERPs.

The systems and methods of the above embodiments may be implemented in a computer system (in particular in computer hardware or in computer software) in addition to the structural components and user interactions described.

The term “computer system” includes the hardware, software and data storage devices for embodying a system or carrying out a method according to the above described embodiments. For example, a computer system may comprise a central processing unit (CPU), input means, output means and data storage. Preferably the computer system has a monitor to provide a visual output display. The data storage may comprise RAM, disk drives or other computer readable media. The computer system may include a plurality of computing devices connected by a network and able to communicate with each other over that network.

The methods of the above embodiments may be provided as computer programs or as computer program products or computer readable media carrying a computer program which is arranged, when run on a computer, to perform the method(s) described above.

The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

In particular, although the methods of the above embodiments have been described as being implemented on the systems of the embodiments described, the methods and systems of the present invention need not be implemented in conjunction with each other, but can be implemented on alternative systems or using alternative methods respectively.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A method for harmonising data from a new machine of a first type with data from a reference machine of a second type, the method comprising:
receiving a new frequency domain spectrum of a process performed by the new machine; and
determining a harmonised new frequency domain spectrum of the process performed by the new machine, the harmonised new frequency domain spectrum being harmonised to a corresponding frequency domain spectrum of the process when the process is performed by the reference machine, by applying a set of harmonising new-machine specific weights to the new frequency domain spectrum, wherein the set of harmonising new machine-specific weights have been determined based on a filter frequency domain spectrum of a hardware filter of the new machine, and a filter frequency domain spectrum of a hardware filter of the reference machine.

2. The method of claim 1, wherein the set of harmonising new-machine specific weights have been determined at least in part by dividing the filter frequency domain spectrum of the hardware filter of the reference machine by the filter frequency domain spectrum of the hardware filter of the new machine.

3. The method of claim 1 or claim 2, wherein:
the filter frequency domain spectrum of the hardware filter of the new machine is an estimated filter frequency domain spectrum of the hardware filter of the new machine; and/or
the filter frequency domain spectrum of the hardware filter of the reference machine is an estimated filter frequency domain spectrum of the hardware filter of the reference machine.

4. The method of claim 3, wherein:
the estimated filter frequency domain spectrum of the hardware filter of the new machine is based on a set of frequency domain spectra for a plurality of different processes performed by one or more machines of the first type; and/or
the estimated filter frequency domain spectrum of the hardware filter of the reference machine is based on a set of frequency domain spectra for a plurality of different processes performed by one or more machines of the second type.

5. The method of claim 4, wherein:
the one or more machines of the first type includes the new machine; and/or
the one or more machines of the second type includes the reference machine.

6. The method of claim 4 or claim 5, wherein:
the set of frequency domain spectra for the plurality of different processes performed by the one or more machines of the first type comprises at least 30 frequency domain spectra; and/or
the set of frequency domain spectra for the plurality of different processes performed by the one or more machines of the second type comprises at least 30 frequency domain spectra.

7. The method of any of claims 4-6, wherein:
the filter frequency domain spectrum of the new machine is based on an average of the set of frequency domain spectra for the plurality of different processes performed by one or more machines of the first type; and/or
the filter frequency domain spectrum of the reference machine is based on an average of the set of frequency domain spectra for the plurality of different processes performed by one or more machines of the second type.

8. The method of any of claims 4-7, wherein the new frequency domain spectrum of the process performed by the new machine is one of the set of frequency domain spectra for the plurality of processes performed by one or more machines of the first type.

9. The method of any preceding claim, further comprising interpolating the harmonising new-machine specific weights across frequency.

10. The method of any preceding claim, wherein the new frequency domain spectrum is received from the new machine, or from local storage.

11. The method of any preceding claim, further comprising:
receiving time series data of the process performed by the new machine; and
transforming the time series data into the new frequency domain spectrum of the process performed by the new machine.

12. The method of claim 11, further comprising retrieving time-domain data from the harmonised new frequency domain spectrum.

13. The method of any preceding claim, wherein the data is electroencephalographic data.

14. The method of any preceding claim, wherein the method is a computer-implemented method.

15. A method for generating a set of harmonising new-machine specific weights for harmonising data from a new machine of a first type with data from a reference machine of a second type, the method comprising:
determining a set of harmonising new machine-specific weights based on a filter frequency domain spectrum of a hardware filter of the reference machine and a filter frequency domain spectrum of a hardware filter of the new machine.

16. The method of claim 15, further comprising:
receiving a set of frequency domain spectra for a plurality of different processes performed by one or more machines of the first type;
determining the filter frequency domain spectrum of the hardware filter of the new machine based on the set of frequency domain spectra for the plurality of different processes performed by the one or more machines of the first type.

17. The method of claim 15 or claim 16, further comprising:
receiving a set of frequency domain spectra for a plurality of different processes performed by one or more machines of the second type; and
determining the filter frequency domain spectrum of the hardware filter of the reference machine based on the set of frequency domain spectra for the plurality of different processes performed by the one or more machines of the second type.

18. The method of any of claims 15-17, wherein the method is a computer-implemented method.

19. A device for harmonising data from a new machine of a first type with data from a reference machine of a second type, the device comprising a processor and a memory, and wherein the memory contains machine executable instructions which, when executed on the processor, cause the processor to:
receive a new frequency domain spectrum of a process performed by the new machine; and
determine a harmonised new frequency domain spectrum of the process performed by the new machine, the harmonised new frequency domain spectrum being harmonised to a corresponding frequency domain spectrum of the process when the process is performed by the reference machine, by applying a set of harmonising new-machine specific weights to the new frequency domain spectrum, wherein the set of harmonising new machine-specific weights have been determined based on a filter frequency domain spectrum of a hardware filter of the new machine, and a filter frequency domain spectrum of a hardware filter of the reference machine.

20. The device of claim 19, wherein the memory contains machine executable instructions which, when executed on the processor, cause the processor to perform the method of any of claims 2-14.
